(19) United States
(12) Patent Application Publication
(10) Pub. No.: US 2013/0072762 A1
(43) Pub. Date: Mar. 21, 2013

(54) APPARATUS AND METHOD FOR ORGANIZING HEALTHCARE MONITOR LEADS

(71) Applicant: Scott Richard SERINO, Alexandria, VA (US)

(72) Inventor: Scott Richard SERINO, Alexandria, VA (US)

(21) Appl. No.: 13/238,599

(22) Filed: Sep. 21, 2011

(51) Int. Cl. A61B 5/00

(52) U.S. Cl. 600/301

(57) ABSTRACT

A method and apparatus for organizing healthcare monitor leads enables the leads to be quickly removed and replaced from their stored positions. Each of the healthcare monitor leads has a closed circular ring attached to the lead typically about 12 to 18 inches from the sensor end. An elongate arm member approximately 4 to 6 inches long is mounted to the monitor boom so that it extends horizontally away from the boom. The monitor leads are stored by hooking each closed circular ring over the elongate arm member so that the leads each hang in a single bight from the monitor to the arm member.

### BACKGROUND OF THE INVENTION

This invention relates generally to hospital equipment and in particular to health care monitors.

Hospital emergency rooms, operating rooms and recovery rooms as well as patient rooms in doctor's offices and other health care facilities are often equipped with patient monitoring devices. These patient monitoring devices typically consist of a video display monitor, which displays the output of various sensors used to monitor patient vital signs. These sensors typically include a pulse oximeter, EKG, blood pressure cuff, thermometer, and other invasive or non-invasive sensors. For each of these sensors there is typically a cable, tube, or other lead (hereinafter “lead”) for transmitting the signal from the sensor to the patient monitoring device. In general, the leads extending from the sensor to the patient monitoring device are from about 4 feet to 12 feet in length and from about 2 mm to 6 mm in diameter.

The extent to which leads attached to the patient monitoring device are organized and stored varies greatly from operating room to operating room and from hospital to hospital. Often, the leads are simply allowed to dangle from the monitor or left on the floor. As a result, the leads tend to get tangled. This creates a cluttered work environment and causes stress to the healthcare providers because the leads must be located and untangled before they can be used which, especially in an emergency room environment, can lead to critical delay in the delivery of healthcare services. Moreover, leads lying on the floor interfere with the movement of hospital gurneys and other equipment as they become entangled in the wheels. Finally, if left on the floor, the leads themselves may become damaged by equipment wheels or as a result of being stepped on by healthcare providers.

U.S. Pat. No. 5,033,474 issued to Varelis et al. discloses a healthcare monitor that includes one or more sensor cables each of which is wound about a spool which prevents the sensor cables from becoming entangled one with another. Although Varelis et al. recognizes the need to provide a means for preventing the sensor cables from becoming entangled one with another, the proposed solution of winding each cable around a spool is cumbersome and therefore unlikely to be practiced, especially in the hectic environment of a hospital emergency room.

U.S. Pat. No. 6,196,503 issued to Cersnosek et al. discloses a healthcare monitor that includes a plurality of sensor cables each of which is coiled and stored on a plurality of hooks mounted to a frame or wall. Although Cersnosek et al. also recognizes the need to provide a means for preventing the sensor cables from becoming entangled one with another, the proposed solution of coiling each lead separately is cumbersome and therefore unlikely to be practiced, especially in the hectic environment of a hospital emergency room.

What is needed and not disclosed in the prior art is an apparatus and method for organizing and storing healthcare monitor leads wherein the leads can be quickly moved from the stored position to the in-use position with minimal effort by the healthcare providers, while still keeping the monitor leads off the floor and out of the way when not in use.

### SUMMARY OF THE INVENTION

The present invention comprises a method and apparatus for organizing healthcare monitor leads in which the leads can be quickly removed and replaced from their stored positions. According to an illustrative embodiment, each of the healthcare monitor leads has a closed circular ring attached to the lead typically about 12 to 18 inches from the sensor end. An elongate arm member approximately 4 to 6 inches long is mounted to the boom on which the monitor is mounted so that the arm extends horizontally away from the boom. The monitor leads are stored by hooking each closed circular ring over the elongate arm member so that the leads each hang in a single bight from the monitor to the elongate arm member. The monitor leads can be removed for use on a patient by sliding the closed circular rings off the end of the elongate arm member. Since the monitor leads are typically no more than 12 feet in length and the monitor boom is typically at least 5 feet above the floor, the single bight is sufficient to prevent the leads from touching the floor at all or very little. In an alternative embodiment, the closed circular rings are replaced with open hooks to enable the monitor leads to be removed individually.

### BRIEF DESCRIPTION OF THE DRAWING

The present invention will be better understood from a reading of the following detailed description, taken in conjunction with the accompanying drawing figures in which like references designate like elements and, in which:

FIG. 1 is a perspective view of an apparatus for organizing healthcare monitor leads incorporating features of the present invention;

FIG. 2 is a perspective view of a healthcare monitor lead for use in connection with the apparatus of FIG. 1;

FIG. 3 is a side view of the arm member portion of the apparatus of FIG. 1; and

FIG. 4 is a perspective view of a healthcare monitor lead having an alternative hanger member.

### DETAILED DESCRIPTION

The drawing figures are intended to illustrate the general manner of construction and are not necessarily to scale. In the detailed description and in the drawing figures, specific illustrative examples are shown and herein described in detail. It should be understood, however, that the drawing figures and detailed description are not intended to limit the invention to the particular form disclosed, but are merely illustrative and intended to teach one of ordinary skill how to make and/or use the invention claimed herein and for setting forth the best mode for carrying out the invention.

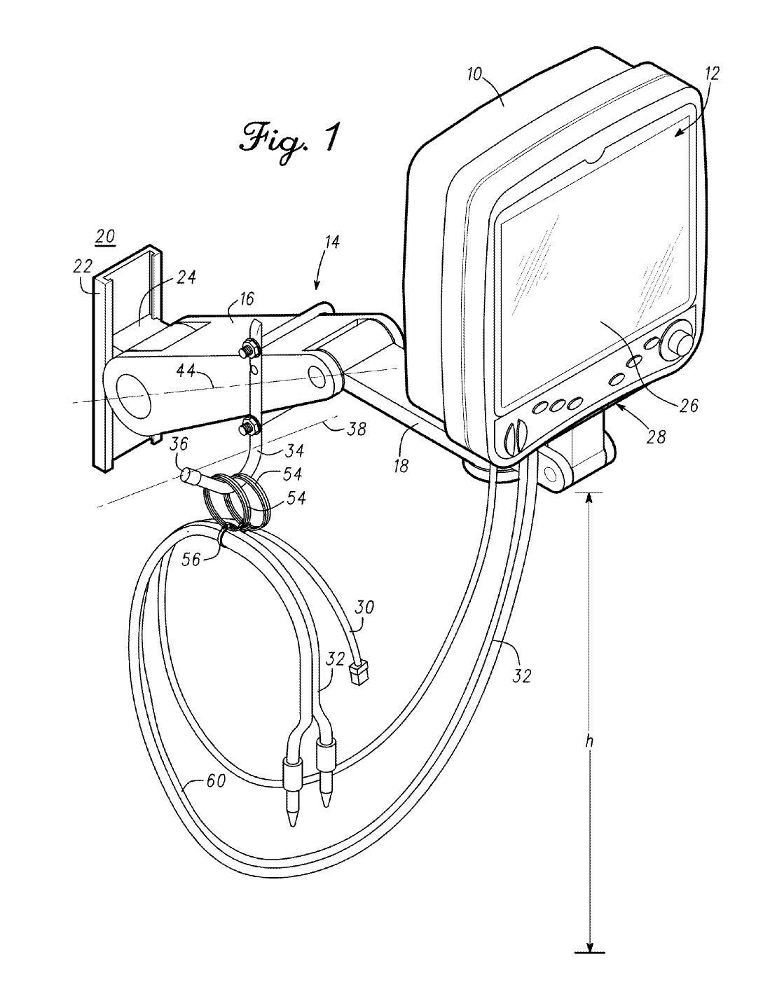

With reference to FIG. 1, a conventional hospital healthcare monitor 10 comprises a video display 12, which is used to display a variety of information to a healthcare provider such as blood pressure, oxygen saturation, heart rate, patient temperature and the like. Display 12 is typically mounted to a boom 14, which may include lower 16 and upper 18 articulating members to enable the height and pitch of display 12 to be adjusted to suit the user. Boom 14, in turn, is typically mounted to a wall 20 or other vertical surface by means of a vertically sliding bracket 22 which enables the base 24 of boom 14 to be raised and lowered. Typically the height (h) of display 12 is adjusted to be at approximately eye-level and the pitch of display 12 is adjusted so that the surface of screen 26 is approximately vertical. Consequently, lower surface 28 of display 12 is typically 5 to 6 feet above the floor.

Healthcare monitor 10 typically includes several leads 30, 32, which may be electrical transducer leads, tubes (e.g. pressure leads to a blood pressure monitoring cuff), or other leads that extend from display 12 (or a nearby processor) to the patient. Absent the method and apparatus disclosed in the present invention, these leads and often the transducers attached to the leads, are simply left to dangle from the monitor 10 onto the floor, where the unsecured ends become entangled and/or damaged by passing traffic.

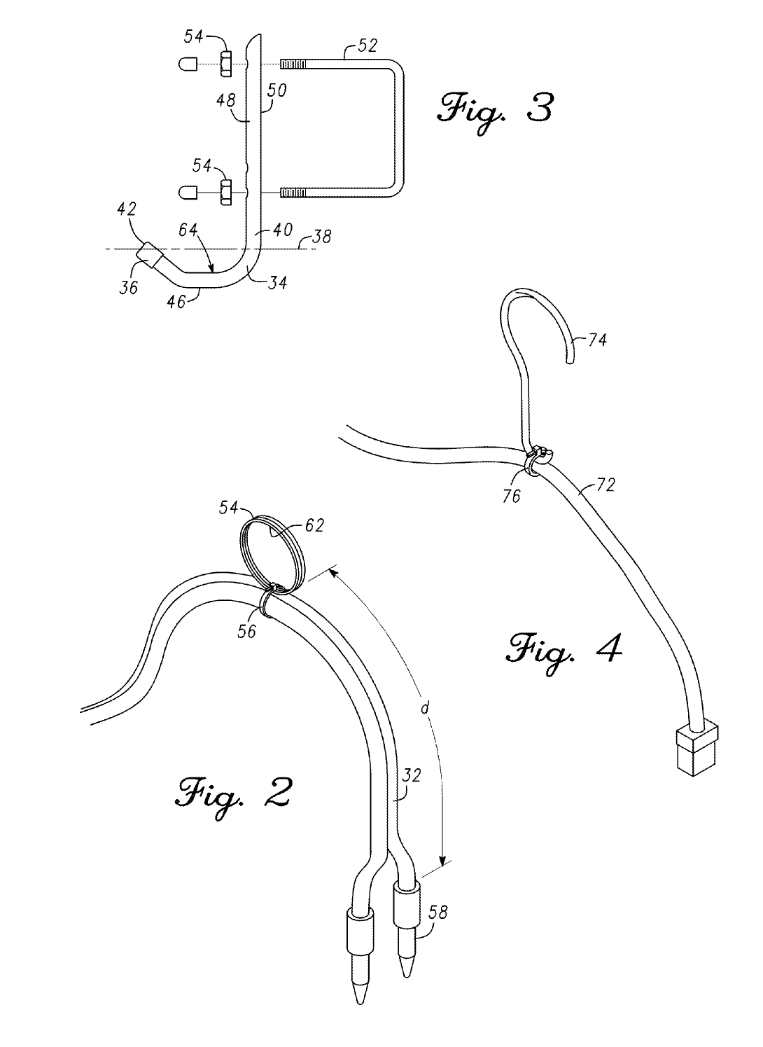

With additional reference to FIGS. 2-4, in accordance with the present invention, an elongate arm member 34 is attached to boom 14 such that the free end 36 of the elongate arm member 34 extends substantially horizontally away from boom 14. As used herein, “substantially horizontally” may include horizontal, upward inclined or a combination thereof such that the general shape of arm member 34 does not promote objects sliding off the tip 36 of arm member 34. Arm member 34 may be smoothly hooked in shape as shown in FIG. 1 or may include a horizontal shank portion 46 alone or in combination with an upturned free end 36 as shown in FIG. 3. In the illustrative embodiment, arm member 34 is mounted so that axis 38 extending through the root 40 and tip 42 of arm member 34 extends substantially perpendicular to longitudinal axis 44 of boom 14, however, any angular orientation between axis 38 and axis 44 that leaves tip 42 of arm member 34 unobstructed is considered within the scope of the present invention.

In the illustrative embodiment, boom 14 has a substantially rectangular cross-section. Consequently, arm member 34 has a vertical shank portion 48 that includes a substantially flat surface 50 adapted to engage and press against a corresponding flat surface of boom 14 to prevent arm member 34 from rotating downward under load. Arm member 34 is secured to boom 14 by means of a U-shaped clamp 52 and conventional hex nuts 54. Although in the illustrative embodiment boom 14 is attached by means of a clamp, any other method of permanent or semi-permanent attachment (e.g. welding, adhesives, sheet metal screws) is considered within the scope of the present invention.

With particular reference to FIG. 2, a hanger member 54 is attached to monitor lead 32 by means of a clamp 56 such that hanger member 54 is held firmly a predetermined distance (d) from the sensor end 58 of monitor lead 32. Clamp 56 may be any conventional hose or tube clamp, cloth loop, hook-and loop fastener, etc. but in the illustrative embodiment comprises a nylon wire zip tie. Monitor lead 32 is stored as shown in FIG. 1 by placing hanger member 54 over arm member 34 so that the lower surface 62 of hanger member 54 engages the upper surface 64 of arm member 34. Typically the distance (d) between hanger member 54 and sensor end 58 is from 12 to 18 inches. Depending on the length of monitor lead 32 and the height of boom 14 above the floor, however, hanger member 54 may be from 12 to 36 inches from sensor end 58 of monitor lead 32 so that monitor lead 32 is suspended in a single bight 60 extending from monitor 10 to arm member 34 with bight 60 suspended above the floor.

In the illustrative embodiment, hanger member 54 comprises a closed circular ring sized to slide easily over free and 36 of arm member 34. For example in the illustrative embodiment, arm member 34 is approximately ½ inch in diameter while hanger member 54 is approximately 1 to 1½ inch in diameter. This enables the monitor leads to be quickly removed and replaced from their stored positions. Although the monitor leads 30, 32 in the illustrative embodiment comprise one single and one dual lead, the present invention is not limited to use with a single and dual lead, but may be used with any number and combination of healthcare monitor leads.

In operation, monitor leads 30, 32 are stored in the position shown in FIG. 1 suspended from arm member 34 by hanger members 54. When monitor leads 30, 32 are to be extended to their in-use configuration monitor leads 30, 32 are quickly slid over the free and 36 of arm member 34. Hanger members 54 are located a sufficient distance (d) from the sensor ends 58 such that hanger members 54 do not interfere with use of the sensors attached to monitor leads 30, 32. Once monitor leads 30, 32 are no longer in use, they are easily replaced to their stored position shown in FIG. 1 with minimal effort by sliding hanger members 54 back over the free and 36 of arm member 34.

As described above, monitor leads 30, 32 (and any other leads) are typically removed in unison for simultaneous use such as in a hospital emergency room when the pulse oximeter, EKG, and blood pressure monitor are used simultaneously. Situations may arise, however, in which various monitor leads are used at different times. Accordingly, as shown in FIG. 4, in an alternative embodiment hanger member 74 comprises an open hook attached to a monitor lead 72 by means of a clamp 76. Because hanger member 74 is an open hook it can be removed and replaced from the side of arm member 34 thereby enabling it to be removed and replaced without disturbing other monitor leads stored on arm member 34. Use of an open hook hanger member 74 also enables arm member 34 to have a closed shape (e.g. a closed rectangle) which may be advantageous in some circumstances.

Although certain illustrative embodiments and methods have been disclosed herein, it will be apparent from the foregoing disclosure to those skilled in the art that variations and modifications of such embodiments and methods may be made without departing from the invention. For example, although in the illustrative embodiment, monitor cords 30, 32 hang directly from the monitor, in certain circumstances it may be advantageous to incorporate a strain relief in the monitor cords proximal the monitor end. To that end, a second hanger member 54, 74 may be attached to monitor cords 30, 32 typically from 12-24 inches from the monitor end of the cord so that the cord hangs in a single bight from the first hanger to the second hanger. Accordingly, it is intended that the invention should be limited only to the extent required by the appended claims and the rules and principles of applicable law. Additionally, as used herein, unless otherwise specifically defined, the terms “substantially” or “generally” when used with mathematical concepts or measurements mean within ±10 degrees of angle or within 10 percent of the measurement, whichever is greater.

What is claimed is:

1. In a health care monitor of the type comprising a display mounted to a substantially horizontal boom, wherein the health care monitor includes a plurality of leads, each of the plurality of leads having a sensor end with a sensor for measuring a clinical parameter of a patient, an improvement comprising:
an elongate arm member having a fixed end, the fixed end being attached to a medial region of the boom, said arm member extending substantially horizontally away from said boom
a plurality of hanger members, each of said plurality of hanger members having a lower surface adapted to engage an upper surface of said elongate arm member for supporting a load suspended from said hanger member, each of said plurality of hanger members being secured to one of said plurality of leads proximal the sensor end thereof;
whereby each of said plurality of leads can be suspended from said elongate arm member by engaging the lower surface of said hangar member with the upper surface of said elongate arm member.

2. The improvement of claim 1, wherein:
said elongate arm member further comprises a free end and each of said plurality of hanger members comprises a closed circular ring.

3. The improvement of claim 1, wherein:
each of said plurality of hanger members comprises an open hook.

4. The improvement of claim 2, wherein:
said elongate arm comprises a substantially horizontally extending portion proximal the fixed end and an upwardly extending portion proximal the free end of said elongate arm.

5. The improvement of claim 1, wherein:
said elongate arm member is attached to said boom by means of a removable clamp.

6. The improvement of claim 5, wherein:
said boom has a rectangular cross section and said removable clamp comprises a U-shaped bolt coupled to a flange, the flange being adapted to engage a flat surface of said rectangular cross section.

7. The improvement of claim 1, wherein:
said elongate arm extends substantially perpendicular to a longitudinal axis of said boom.

8. The improvement of claim 1, wherein:
each of said plurality of hanger members is attached to a corresponding one of said plurality of leads between 4 and 24 inches from the sensor end of said lead.

9. The improvement of claim 1, wherein:
said boom is arranged so that said arm member is located predetermined distance above a floor surface, wherein each of said plurality of hanger members is attached to a corresponding one of said plurality of leads a sufficient distance from the sensor end such that said plurality of leads are each suspended in a single bight.

10. A method of organizing a plurality of elongate sensor leads attached to a health care monitor having a display mounted to a boom, the method comprising:
providing a substantially horizontal elongate arm member having a fixed end, the fixed end being attached to a medial region of the boom;
providing a plurality of hanger members, each of said plurality of hanger members having a lower surface adapted to engage an upper surface of said elongate arm member for supporting a load suspended from said hanger member, each of said plurality of hanger members being secured to one of a plurality of sensor leads proximal the sensor end thereof;
suspending said plurality of sensor leads proximal the sensor ends thereof by hanging said hanger members from said elongate arm member; and
removing at least one of said plurality of hanger members from said elongate arm member in order to measure a clinical parameter of a patient.

11. The method of claim 10, wherein:
each of said plurality of hanger members is attached to a corresponding one of said plurality of leads a sufficient distance from the sensor end such that said plurality of leads are each suspended in a single bight that does not touch the floor surface.

12. The method of claim 10, wherein:
said elongate arm member has a free end and wherein said plurality of hanger members are removed by sliding said plurality of hangar members off the free end of said elongate arm member.
